(19) United States
(12) Patent Application Publication
(10) Pub. No.: US 2011/0229584 A1
(43) Pub. Date: Sep. 22, 2011

(54) Compound for the control of herpes simplex virus using glycyrrhizic acid, lipoic acid, allantoin, and slippery elm

(76) Inventor: James David Burrell, Pittsboro, NC (US)

(21) Appl. No.: 12/661,462

(22) Filed: Mar. 17, 2010

(51) Int. Cl. A61K 33/30; A61P 31/22; A61P 31/20; A61P 1/04; A61P 1/00; A61P 29/00

(52) U.S. Cl. 424/642; 424/641

(57) ABSTRACT

A Theraputic composition of glycyrrhizic acid, slippery elm, zinc oxide, allantoin, lysine monohydrochloride, L-Carnitine, Lipoic acid, Salicylic Acid, Citric Acid, vitamin E Acetate, wheat germ oil, and Shea butter for the treatment of HSV-1, HSV-2, canker sores, shingles, and other epidermal and oral ailments.

### BACKGROUND OF THE INVENTION

1. Field of the Invention

The present invention relates to treatment for HSV-1, HSV-1 related ailments that treats both the root HSV-1 virus while synergistically alleviating the symptoms of HSV-1 and HSV-1 related ailments by soothing general oral irritations, promoting oral health, working against oral lesions, and blocking the sun. Furthermore, it relates to a topical treatment for aphthous ulcers (canker sores), and Herpes Zoster (shingles), and general epidermal and oral discomfort. The pro-health qualities of the present invention render it useful in helping cancer patients recover from aggressive therapies that may leave them vulnerable to oral and pharyngeal disorders.

2. Descriptions of Prior Art

U.S. Pat. No. 3,806,617, issued to Smiley discloses a process for the preparation of licorice candy

U.S. Pat. No. 5,362,737, issued to Vora, et al., discloses salicylic acid as a treatment for aphthous ulces.

U.S. Pat. No. 5,534,554, issued to Katz discloses a method of treatment of epidermal viral disorders, including HSV-1, HSV-1 Zoster, and Aphthous ulcer, by administering topically a compound with n-Docosanol.

U.S. Pat. No. 5,643,584, issued to Sintov, et al. discloses the topical composition Acyclovir for the treatment of viral epidermal disorders, including Aphthous ulcer, HSV-1, HSV2, and Zoster.

U.S. Pat. No. 5,660,871, issued to Schantz, et al. discloses a method for producing a nonblocking, free flowing licorice extract.

U.S. Pat. No. 6,977,081, issued to Rood et al., discloses epidermal effects of allantoin.

U.S. Pat. No. 6,124,304, issued to Boon et al., discloses penciclovir for the treatment of zoster associated pain via topical administration and famciclovir administered orally for the treatment of zoster associated pain.

U.S. Pat. No. 7,632,662, issued to Pettigrew et al., discloses the antioxidant properties of L-caniitine.

European Patent EP05330 discloses penciclovir and famciclovir and related ganine derivatives as topical and orally administered treatment of HSV-1 related diseases.

In this respect, the therapeutic composition for the treatment of HSV-1 and HSV-1 related disorders, according to the present invention, substantially departs from the conventional concepts and designs in the prior art and in doing so provides a composition primarily developed for the purpose of treating HSV-1 and HSV-1 related disorders.

Therefore, it can be stated that there exists a continuing need for a new and improved therapeutic composition for the prevention and treatment of HSV-1, HSV-2, Herpes Zoster, and aphthous ulcers which can be used for treating HSV-1, HSV-2, Herpes Zoster, aphthous ulcers and related maladies with a new combination of therapeutic agents. In this regard, the present invention substantially fulfills this need.

### SUMMARY OF THE INVENTION

In view of the foregoing disadvantages inherent in other methods of treating HSV-1, HSV-2, Herpes Zoster, and aphthous ulcers in prior art, the present invention provides an improved therapeutic composition for the prevention and treatment of oral disorders associated with the aforementioned viruses

To attain this, the present invention supplies a synergistic combination of compounds designed to treat the virus itself as well as to alleviate the symptoms and triggers associated with HSV-1, HSV-2, Herpes Zoster, and aphthous ulcers.

First provided is glycyrrhizic acid. This is a derivative of roots of the licorice plant. It is the sweet tasting compound from the root. Glycrrhizic is extracted from Glycyrrhiza plants specifically Glcyrrhiza Glabra, G. Kanseusis, and G. inflata.

Next provided is Zinc oxide. Zinc oxide is produced synthetically.

Next provided is the herb Slippery elm (Ulmus rubra). The slippery elm used comes from the inner bark, which is either dried and made into a powder or moistened with water.

Next provided is the chemical compound Allantoin. Allantoin is a naturally harvested compound by many organisms, including animals, plants, and bacteria, or is synthetically produced.

Next provided is L-Lysine Monohydrochloride. L-Lysine Monohydrochloride is an essential amino acid. It is biosynthesized.

Next provided is Carnitine. Carnitine is a quarternary ammonium compound biosynthesized from the amino acids lysine and methionine.

Next provided is alpha-Lipoic acid. Alpha-lipoic acid is an organosulfur that is essential for aerobic life. It is biosynthesized.

Next provided is salicylic acid. Salicylic acid is a beta hydroxyl acid obtained from the bark of the willow tree of any member of the Salix genus.

Next provided is citric acid. Citric acid is a weak organic acid. Citric acid can be biosynthesized or extracted from citrus fruits

Next provided is vitamin E acetate. Vitamin E acetate, or tocopherol acetate, is a common vitamin supplement that is synthesized.

Next provided is wheat Germ oil. Wheat germ oil is extracted from the germ of the wheat kernel.

Next provided is shea butter. Shea butter is a natural fat derived from the seed of the shea tree (butyrospermum parkil).

The invention is capable of other embodiments and is capable of being practiced in various ways. It is, furthermore, to be understood that the phraseology and terminology here are for descriptive purposes only and should not be read as limiting.

As such, those skilled in the art will understand that the conception upon which this disclosure is based is may be readily utilized in executing various designs, embodiments, and applications many of which would be able to execute the many utilities of the present invention. It is important, then, that it be understood that the claims made are done so to include such constructions so long as they do not exceed the scope of that which is claimed in the invention.

It is an object of the present invention to facilitate the provision of a new composition and delivery system for the treatment of HSV-1, HSV-2, apthous ulcers, Herpes Zoster related, and other related oral epidermal disorders.

Another object of the present invention is to facilitate the provision of a new composition and delivery system for the treatment of HSV-2 and HSV-2 related oral disorders.

Another object of the present invention is to facilitate the provision of a new composition and delivery system for the treatment of herpes zoster and herpes zoster related disorders.

Another object of the present invention is to facilitate the provision of a new composition and delivery system for the treatment of aphthous ulcers and aphthous ulcer related disorders.

Another object of the invention is to provide a new and improved therapeutic composition and treatment delivery system that is easily manufactured.

Another object of the invention is to provide a new and improved therapeutic composition with a new combination of therapeutic agents.

Another object of the invention is to provide a new therapeutic composition including glycyrrhizic acid, allantoin, slippery elm, zinc oxide, Lysine Monohydrochloride, L-Carnitine, Lipoic acid, Salicylic Acid, Citric Acid, vitamin E Acetate, wheat germ oil, stevioside, and Shea butter.

Another object of the present invention is to provide a topical delivery system that is easily transportable and stored for use by the public.

### DESCRIPTION OF THE PREFERRED EMBODIMENT

The preferred embodiment of the new and improved composition for the treatment of HSV-1, HSV-1 related oral disorders, HSV-2, aphthous ulcers, and herpes zoster employing the principles and concepts of the present invention, will be described.

The present invention, the composition for the treatment of HSV-1, HSV-1 Related oral disorders, HSV-2, Herpes Zoster, aphthous ulcers, and general oral maladies, is composed of a group of components, none of which comprise a majority of a product. Those components include slippery elm, glycyrrhizic acid, allantoin zinc oxide, Lysine Monohydrochloride L-carrnitine, Lipoic acid, Salicylic Acid, Citric Acid, vitamin E Acetate, and wheat germ oil.

These components are measured as individuals and then put into practice as a whole in order to achieve the optimal treatment of the disease.

The desired result is a composition that is provided as therapy for the treatment of HSV-1, HSV-1 related disorders, HSV-2, Herpes Zoster, aphthous ulcers, and other maladies of the lips. The composition is designed such that it has a solid ointment form and can be applied topically to the entire surface area of the lips.

First provided is glycyrrhizic acid. Glycyrrhizic acid and its derivatives have many noted biotheraputic effects. It is known as an antiviral agent, has been shown to have qualities that fight ulcers and it also has anti-inflammatory effects.

Glycyrrhizic acid is a triterpenic component of Glycyrrhizia glabra roots has been proven to have antiviral action. Salts of glycyrrhizic aid are widely used as sweeteners in candy, tobacco, and toothpaste. Glycyrrhizic acid consists of one molecule of glycrrhetic acid, which is the pharmacologically active component, and two molecules of glucuronic acid. These compounds are obtained by hydrolysis of glycyrrhizic acid by beta-glucoronidase. Glycyrrhizic acid exists in two stereoisomeric forms, 18-beta and 18-alpha. It has been shown to both inhibit the growth and that cytopathic effect of vaccinia, Herpes simplex (HSV), Newcastle's Disease, and vesicular viomantitis. Furthermore, it has been proven to irreversibly deactiveate HSV. (http://www.springerlink.com/content/p2n76p0k36878412).

Dried, aqueous extracts from the root of G. glabra contain 10-20% glcyrrhizic acid. Experiments in humans show that intestinal bacteria hydrolyze glycyrrhirizic acid into glycyrretic acid.

Furthermore, the same glycyrrhizic acid derived from licorice root is known to have a high anti-inflamatory component without many of the side effects proven to exist in many of the NSAID anti-inflammatory treatments (http://resources.metapress.com/pdf-preview.axd?code=9lxle4wn7f2g16x8&size=largest). Furthermore, it has been documented that both glycyrhyzic acid in its trans form, as is most commonly used in cosmetics, but also many of the esters of glycyrrhizic acid have proven antiulcer activity. The document will later show that in addition to being able to permanently neutralize the activity of the virus, the glycyrrhizic acid is useful because irritation and ulcers are common symptoms associated with HSV-1 infections.

Furthermore, the anti-inflammatory effects support general oral and phyrangeal health in a way that will assist cancer patients during recovery.

Methods for the processing of licorice extract are disclosed in U.S. Pat. No. 5,660,871, issued to Schantz, and U.S. Pat. No. 3,806,617, issued to Smylie et al.

Glycyrrhizic acid and glycrrhetic acid were found to inhibit severe acute respiratory syndrome (SARS) (Cinatl et al) Glycyrrhizic acid was proven to improve the resistance of thermally injured mice to HSV-1. Glycyrrhizic acid was found to show strong activity against in vivo HSV-1.

Delphour A. R. et al. (J Pharm Pharmacol 1994 February; 46( ):149-9) reported protective effects of licorice derivatives against ulcers. This supports both viral treatments and cancer recovery by protecting the mouth and throat.

Glycyrrhizin has been shown to be effective against varicella zoster and HSV-2. In studies it was shown that glcyrrhizin inactivates more than 90% of zoster, Epstein-Barr and HSV-2 when incubated with the viruses for more than 30 minutes. Furthermore, glycyrrhizin is shown to be effective against hepatitis, HIV, Corona virus, cytomegalovirus, and upper respiratory tract infections (Fiore C, Eisnhut M, Krausse r et al. Antiviral effect of Glycyrrhia species. Phytother. Res. 2008; 22:141-14, Numazaki K. Glycyrrhiin therapy for viral infections. African j. Biotech. 2003; 210):392-393.).

Next provided is Zinc oxide. Zinc is first known to be able to protect against both sunburn and wind burn and is known to have natural sun blocking properties that decrease the ability of ultraviolet radiation. (Emsley, John (2001). “Zinc”. Nature's Building Blocks: An A-Z Guide to the Elements. Oxford, England, UK: Oxford University Press. pp. 499-505. ISBN 0198503407. http://books.google.com/books?id=j-Xu07p3cKwC). Zinc oxide is sed as a transparent broad-spetrum sunbloc that can attenuate UV radiation throughout the UV spectrum. It has found to be photostable and does not react with organic sunscreens under irradiation (J Am Acad Dermatol 1999; 40; 85-9.0) As will be demonstrated later, exposure to the sun is one of the triggers of HSV-1 diseases. Furthermore, both sunburn and windburn are general maladies of the lips that affect even those not afflicted with HSV-1. The disclosed therapeutic compound and delivery system would be able to assist in the prevention of common malady which affects the lips and epidermis.

Zinc oxide is known to have anti-oxidant properties (http://jn.nutrition.org/cgi/content/abstract/130/5/1447S). Anti-oxidant properties are capable of retarding the process of aging and oxidation in human skin, thus providing a pro-health element to the active ingredients of the new and improved therapeutic composition and delivery system.

Next provided is slippery elm. Slippery elm is known to have demulcent properties (http://patft.uspto.gov/netacgi/nph-Parser?Sect1=PTO2&Sect2=HITOFF&p=1&u=%2Fnetahtml%2FPTO%2Fsearch-bool.html&r=1&f=G-&1=50&co1=AND&d=PTXT&s1=%22slippery+elm%22&s2=demulcent& OS=%22slippery+elm%22+AND+demulcent&RS=%22slippery+elm%22+AND+demulcent). Demulcents have the ability both to soothe whatever irritants in the mucous membrane are in place and to motivate the mucous membrane. They provide a protective film over the surface of the mucous membrane that helps protect from all forms of maladies and also serves to soothe and caress the membrane.

U.S. Patent 20,090,324,734 application discloses slippery elm as an emulsant. Emulsants assist in the soothing process or discomfort and they are naturally hydrophilic and can attract moisture to areas that are dry as a result of herpes sores.

Slippery Elm is known to have anti-ulcer properties. http://patft.uspto.gov/netacgi/nph-Parser?Sect1=PTO2&Sect2=HITOFF&p=1&u=%2Fnetahtml%2FPTO%2Fsearch-bool.html&r=2&f=G&l=50&co1=AND&d=PTXT&s1=%22slippery+elm%22&s2=ulcer&OS=%22slippery+elm%22+AND+ulcer&RS=%22slippery+elm%22+AND+ulcer). Some of the lesions formed on the mouth as a result of Oral HSV-1 \ are ulcers and this property will help to alleviate some of those symptoms of HSV-1 as well as any other oral malady whose symptoms include ulcers.

Next provided is allantoin. Allantoin is a white, odorless substance derived biosynthetically from uric acid. It can come from many sources. U.S. Pat. No. 6,977,081, issued to Rood, discloses allantoin's ability to assist skin cell regeneration, its keratolytic effects. Allantoin is non toxic, non-allergenic, and it has anti-inflammatory properties disclosed here as well. HSV-1, HSV-2, Zoster, and aphthous ulcer related diseases diseases often have as symptoms that include lesions.

Allantoin is known to have anti-irritant effects. (http://patft.uspto.gov/netacgi/nph-Parser?Sect 1=PTO2&Sect2=HITOFF&u=%2Fnetahtml%2FPTO%2Fsearch-adv.htm&r=1&f=G&l=50&d=PTXT&p=1&p=1& S1=(allantoin+AND+anti-irritant)&OS=allantoin+AND+anti-irritant&RS=(allantoin+AND+anti-irritant)). Many maladies of the epidermal layer of the lips as well as HSV-1 and HSV-1 related infirmities have the effect of causing irritation. Allantoin will help alleviate irritation associated with these maladies as well as action against the kinds of disorders common in the aftermath of aggressive cancer therapies.

Next provided is lysine monohydrochloride. Lysine monohydrochloride has been proven not to be able to inactivate HSV-1 when applied topically to infected epidermal areas but rather has been proven to have the capacity to control the symptoms of HSV-1 and reduce the occurrence of reactivated HSV-1.

Next provided is L-Carnitine. Carnitine is added as an antioxidant. Antioxidants help prevent lipid peroxidation of phosopholipid membranes and against oxidative stress induced at the endothelial and myocardial cell level. Reducing oxidative stress helps defend the body against any ailment in which tissue damage is a cause or effect as oxidative stress is a cause of the much of this damage. In the case of HSV-1, the sores that accompany infection damage the epidermal layer of the lips. This tissue damage is mitigated by the antioxidant effects of Carnitine.

L-Carnitine has been proven to restore the energy production of mitochondria. Mitochondria are membrane-enclosed organelles found in all cells. They generate the energy used to fuel many of the cell's processes. They are involved in the control of the cellular cycle and cellular death. Extending the ability of mitochondria to produce cellular energy can extend the life of cells.

Next provided is Alpha-lipoic Acid. U.S. Pat. No. 7,632,662 discloses the use of Lipoic acid as an antioxidant. Lipoic acid is included for its antioxidant effects and provides the same antioxidant effects described, supra, by L-carnitine.

Lipoic acid has anti-inflammatory qualities. This occurs because the phosphoinositide 3-knase/protein kinase signaling pathway was shown to increase LPS-induced inflammatory responses. The Lipoic acid was able to inhibit the LPS-caused expression of the cellular adhesion molecules and the adherence of monoctes to the human aortic endothelial cells. Lipoic acid caused an increase in the phosphorlation of the protein inane and inhibited the lPS-triggered NF-B DNA binding activities. This attenuation proved that lipoic acid is useful in shortening the period of certain inflammations, such as those associated with HSV.

Alpha-lipoic acid has been found to restore cells and has been shown to have activity protecting mitochondria from free radicals. This property has activity against all forms of disorders related to mitochondrial deletion and dysfunction. In the present invention, the alpha-lipoic acid would help prevent cellular death in the aftermath of radiation and chemo-therapy.

Next provided is salicylic Acid. U.S. Pat. No. 3,682,968 discloses salicylic acid's anti-inflammatory properties. The epidermal viral infections associated with HSV, Zoster, and aphthous ulcers all have as part of their symptoms inflamed lesions of the epidermis

U.S. Pat. No. 5,362,737 discloses salicylic acid as having activity against pain associated with aphthous ulcers. One of the goals of the present composition is to further the treatment of aphthous ulcer. The activity against aphthous ulcer pain furthers those treatment goals.

U.S. Patent Application No. 20,090,232,756 discloses keratolytic and anti-aging properties of salicylic acid. As discussed, supra, many viral epidermal disorders can cause roughness of skin and the eratolytic effect of salicylic acid can assist this problem.

Next provided is Vitamin E Acetate. EU Patent No. 0,910,367 discloses Vitamin E Acetate as being effective as a moisturizing agent and a protecting agent that shields the skin in order to support healing. It is known to support the treatment of skin xerosis, contact dermatitis, cheilitis, keratosis, psoriasis, burns, herpes, skin erosions, insect stings or bites, skin ulcers, bed sores, proctitis, hemorrhoids, eczema, vestibulitis, and other maladies of the skin

Next provided is wheat germ oil. U.S. Patent Application No. 20,902,085 discloses wheat germ oil as an antioxidant.

Next provided is stevioside. Stevioside is provided as a sugar-free sweetener that improves the taste of the present composition without exposing any diabetic patients to the risks associated with sugar consumption.

Next provided is raspberry. Raspberry is provided as a flavor agent in order to improve the flavor of the present composition.

Oral HSV-1 (better known as Oral Herpes) manifests itself as sores or blisters. It is most commonly spread via skin to skin contact with the infected area. It can also spread via shared food or drink. Before the blister appears, the area where it will appear may feel inflamed. The pain may occur two days before the blister appears. The sore will then appear as a small fluid blister. It will normally weep (ulcerate) and form a scab before healing. This process takes from 8 to 14 days. Once one has contracted HSV-1, it remains dormant in them until neutralized and can be triggered in many different ways. It has solar triggers, stress, dietary, fatigue, skin irritation, and climate triggers (such as extreme heat or cold). Fever can trigger it and so can random ultraviolet light exposure.

HSV-1 is one of herpes virus group. Herpes viruses are DNA viruses with double-stranded DNA genomes. Herpesviridae viruses infect their hosts with an initial infection and then establish latent infections in the body. They are initially contracted due to external interaction with the virus. The viruses are then re-activated due to external factors which cause weakness in the immune systems of the human beings. Humans who suffer from viral infections often suffer recurrence. The symptoms of these viruses include rashes, pain, and open sores. Herpes sores on the oral epidermal layer can often cause major social discomfort. Furthermore, the existence of open sores often leaves the afflicted individual susceptible to bacterial infections

HSV-1 is transmitted by an individual shedding the virus coming into contact with another individual. HSV infection occurs via interaction with glycoproteins on the envelope of the virus with receptors on the surface of the cell. The viral envelope will then create a pore in the surface of the cell by triggering the cell's natural capacity to become selectively permeable in order to permit certain outside particles into its wall. The complementary receptors attract the virus and the cell to each other, causing them to merge. HSV uses glycoprotein C in its envelope in order to attach to the cell. Glycoprotein C interacts with the cell's receptor heparin sulfate. Furthermore, glycoprotein D bonds with the herpes virus mediator receptor. Once this bond has been formed, other glycoproteins in the virus are able to interact with components within the cell. Once this has occurred, glycoprotein d changes its conformation and interacts with glycoprotein and glycoprotein D to form a complex. Glycoprotein B then interacts with this process to form a functional pore that permits the capsid of the virus to enter into the cell. Once this has occurred, the cell is infected with HSV-1.

There are currently two topical products for the specific topical treatment of oral HSV-1. The treatment of oral HSV-1 often includes the combination of existing topical antibiotics for the treatment of bacterial infections with oral antiviral medications including but not limited to Acyclovir, Valacyclovir, and Famcyclovir. Furthermore, those afflicted with HSV-1 are often directed to reduce their exposure to the sun and to take pain medication such as ibuprofen and aspirin in order to alleviate the pain they feel.

Abreva is an over the counter topical medication designed and approved to treat HSV via topical application. Abreva is an over the counter medication that employs docosanol (or behenyl alcohol) to fight against HSV. Abreva is presumed to work by inhibiting the fusion of the human host cell with the viral envelope, thus preventing HSV to infect cells in the treated area. Abreva was found to shorten the healing time of the infection in a trial by 17.5 hours. It was also found that claims that treatment with Abreva cut the recovery time in half were misleading. The product claimed in the present application operates differently from how Abreva is presumed to work. It employs its principal active ingredient, glycyrrhic acid, in order to actually deactivate HSV. This has been proven as incontrovertible scientific fact by lab studies. As will be discussed later, the product clamed in the present invention also provides a synergistic component by which its various other ingredients assist to alleviate the symptoms of HSV-1 and help prevent the triggers that cause recurrence of HSV. The present claimed invention provides an improved and more comprehensive treatment of HSV and HSV related symptoms.

In clinical trials showing the healing time associated with the use of docosanol, Docosanol was found to reduce the healing times of first episodes b an average of 1.9 days from 7.3 days to 5.4 days. In tests done in instances in which the treatment was applied to a crossover study group, healing time was found to decrease from an average of 8 days Furthermore, docosanol was found to decrease the mean time of healing by more than 50% in both treatment phases when applied early in the stages of infection.

Penciclovir is also a topical cream that has been approved for the treatment of HSV-1. Penciclovir is primarily a vehicle for the healing of the classical lesions, reducing the pain felt by patients, and ceasing the shedding of the virus. In a randomized, double blind study, it was found that the healing of the lesions was 0.7 days (5.5 days decreased to 4.8 days) faster for those who were treated with penciclovir versus placebo, pain duration was decreased by 0.6 days (from 4.1 days to 3.5 days), and lesion virus shedding was marginally changed.

The results of the studies on Penciclovir and docosanol establish a standard for what treatments have been deemed acceptable as useful in the treatment of HSV-1 in the form of herpes simplex labialis or cold sores. Furthermore, the fact that these topical treatment options still leave considerable amounts of time during which those afflicted suffer demonstrates a continuing need for improved treatment.

Many of the orally ingested treatments have reported as side effects vomiting, fatigue, headache, dizziness, and stomach pain. There is a need to create treatments with compounds that are as natural as possible in order to minimize side effects.

While these devices and methods fulfill their respective, particular objectives and requirements, the aforementioned patents do not describe therapeutic composition for the treatment of HSV-1 and HSV-1 related disorders that allow treating HSV-1 with a new combination of therapeutic agents released.

It is therefore, an object of the invention to provide improved treatment for oral HSV-1 that both treats pain and acts as an antiviral.

It is further an object of the invention to provide an improved treatment for oral and pharyngeal surfaces adversely affected by the disease.

It is further an object of the invention to provide an improved comprehensive treatment that can reconstruct the epidermal layer of the mouth and the surface layer of the throat when any form of lesion has been suffered.

It is further an object of an invention to treat HSV-1 with natural compounds.

It is further an object of this invention to provide components in dosages suitable for treatment uses.

HSV-2 is virtually identical to HSV-1 with the principal difference being their preferred zones of infection. HSV-1 prefers the oral regions of the body whereas HSV-2 is principally a genital disease. HSV-2 is commonly transferred as neonatal herpes, which can be dangerous. HSV-2 can be characterized by as little as no noticeable symptoms to tingling and numbness to painful genital ulcers, painful urination, fever, and headaches. HSV2 can reactivate from its dormant state. This can result in viral shedding, which can last 1-5 days. Anti-viral medications currently can help shorten and prevent outbreaks. Daily suppressive therapy can reduce transmission. The present disclosed composition has action against the ulcers and the epidermal shedding associated with HSV-2.

HSV-2 is currently treated with dynamiclear as a topical product. It functions by applying the actions of sulfur and St. John's Wort. It can kill the local areas of Herpes. The only noticeable side effect is a moderate stinging pain at the time of the application.

As with HSV-1, oral and topical antivirals including Famvir, Acyclovir, and valacyclovir are effective in reducing the duration of outbreaks. Antivirals can treat outbreaks as the happen or prevent or delay recurrent outbreaks. The most commonly reported side effects of these treatments are headache, nausea, diarrhea, and abdominal pain. Other side-effects may include vomiting and fatigue. Furthermore, there have been instances of serious complications associated with antiviral treatments in which the afflicted patient is suffering from a compromised immune system such as from kidney complications or AIDS.

It is, therefore, a further to provide an improved treatment that both suppresses HSV-2 and provides episodic treatment.

It is further an object of the invention to provide an improved treatment for HSV-2 without the side effects currently associated side effects.

It is further an object of the invention to provide an improved treatment for HSV-2 that will both treat the virus directly and support general epidermal health in order to reduce the symptoms.

Aphthous ulcers, or canker sores, are ulcerations on the mucus membranes. They are often triggered by viral infections. They can be distinguished from other viral oral ulcerations by their stronger tendency to reoccur. Larger aphthous ulcers also occur quite often on non-keratinizing surfaces. Aphthous ulcers may cause fever, swelling of the lymph nodes, irritation and pain on the affected areas. Aphthous ulces may be caused by citrus fruits, physical trauma, weight loss food allergies, chemotherapy, and oral trauma (Lewkowicz N, Lewkowic P, Banasik M, Kumatowska A, Tchorzeski H, (2005). “Predominance of Type 1 cytokines and decreased number of CD4(+)CD25(+high T regulatory cells in peripheral blood of patients with recurrent aphthous ulerations”. Immunol Lett. 99 (1): 57-62. doi: 10.1016/j.imlet.2005.01.001 PMID 15894112, Way D, Ferguson M, Hutheon W, Dagg J)198), :Nutritious deficiencies in recurrent apthae”. J Oral Pathol 7(6): 418-423. Aphthous ulcers have also been linked to Crohn's disease and malnutrition. (Current Medical Diagnosis Treatment 2007, Forty-Sixth Ed (200), Edited by McPhee, S J. MD, Papadakis, M A MD and Tiern, L M, J, MD with Associated Authors—The McGraw-Hill Companies, Inc, New York, USA). Viral aphthous ulcers can be caused by herpesvius, cytomegalovirus, varicella, and coxsackievirus. Treponemal aphthous ulcers can be caused by syphilis. Fungal aphthous ulcers can be caused mucomycosis, histoplasma, and cryptosporidium. Autoimmune aphthous ulcers an be caused by Lupus, Behget's syndrome, Relter's syndrome, inflammatory bowel disease, bullous pemphigoid, and pemphigus vulgaris. Hematologic aphtous ulcers can be caused by cyclic neutropenia. Neoplasmic aphthous ulcers can be caused by squamous cell carcinoma. Currently, ingredients disclosed in the present composition used in the treatment of apthous ulcers include L-Lysine and licorice root. Furthermore, prescription treatments common include steroids and antivirals.

Antibiotic treatments can be used to treat aphthous ulcer. Tetracycline and minocycline are the most commonly used via tablets dissolved in water. Local antiinflamatory agents can be used for the treatment of mnor aphthous ulcers. These include but are not limited to triamcinoclone. Immunomodulators that increase the immune system's ability to suppress aphthous ulcers are also used in the treatment of aphthous ulcers. Thalomide is the most frequently used immune modulator for the management of aphtous ulcers. Aphthasol has been shown to also function as an immunomodulator with action against aphthous ulcers. Analgesics and anti-inflammatory treatments including but not limited to benzocaine, Orabase, and Zilactin-B. Sometimes silver nitrate is employed tough this application is painful and ma cause necrosis and delayed healing. Magnesium hydroide and diphenhydramine hydrochloride mixed brings some symptom relief. inc gluconate and natural agents including but not limited to vitamin C, vitamin B, sage, and echinaccea have often been used to alleviate some symptoms of aphthous ulcer.

It is an object of the present invention to provide a new therapeutic composition for the treatment of aphthous ulcers.

It is further an object of the present invention to provide a therapeutic composition for the treatment of aphthous ulcers that combines antiviral, anti-inflammatory, anti-pain, pro-immune system formula that helps the treatment of aphthous ulcers.

Herpes zoster, or shingles, is a viral disease characterized by painful rash and blisters. Shingles is caused by the varicella zoster virus (VZV), the same virus that causes chicken pox. Shingles occurs when a person who has been infected with chicken pox suffers a reactivation of the varicella zoster virus in their nerves cells, from whence the virus goes to the epidermal layer causing a painful rash. Furthermore, fever and general malaise are associated with zoster. Also, ocular difficulties are often associated with zoster. Zoster is a double-stranded DNA virus and is related to HSV. It remains latent in those who have suffered chickenpox in the ganglia adjacent to the spinal chord or the gaglion semilunare. True latency has not been established as it has been proven that VSV continues to reproduce after chicken pox has been suppressed by the immune system. Unless the immune system is compromised, the system will suppress VSV. It is when this compromise occurs that zoster occurs. While Zoster itself cannot be spread from person to person, VZV can be spread to someone never infected via contact with an individual infected with Zoster. Currently, antivirals and analgesis are administered in the treatment of zoster. Valacyclovir and famciclovir have been proven effective both as treatment and as prophylaxis.

Steroids are often prescribed in the recovery process. Normally zoster infections have a duration of two to four weeks.

It is an object of the present invention to supply a composition of ingredients not yet used in the treatment of zoster that combines anti-viral effects, anti-inflammatory effects, pro-immune system activity.

It is further an object of the present invention to carry out this treatment activity with natural ingredients.

It is further an object of the present invention to treat zoster without the side effects currently associated with oral antivirals.

Virtually all chemotherapy and radiation has among its side effects immunosuppression and myelosuppression. This often comes as a result of paralysis of the bone marrow. It leads to the decrease of red blood cells and white blood cells and it decreases the ability of the body to be able to defend itself from otherwise common infections. Furthermore, circulating endothelial cells in the aftermath of chemotherapy demonstrate the endothelial damage done by chemotherapy. This endothelial damage subjects patients to yeast and bacterial infections. The pro-health effects of the present disclosure support general oral and pharyngeal health and would serve to assist patients in their recovery time from chemotherapy and could reduce some of the endothelial damage caused by chemotherapy if used as a prophylactic. The predominant manifestation of this damage is mucositis, which occurs in virtually all instances of chemotherapy and radiation therapy related to cancers of the head and neck. Mucositis is the painfl inflammation and ulcerations lining the digestive tract. Mucositis affects its victims in 5 stages. Different cytokines are responsible for the various stages of the affliction. Signs and symptoms include swollen mouth and gums, blood in the mouth or throat, difficulty swallowing, soft whitish patches or pus in the mouth or on tongue, sores in the mouth or on the gums or tongue, and increased mucous or thicker saliva in the mouth.

Treatments against mucositis include allpurinol and cyotherapy. Mouthwashes are often administered that include cortiosteroids, benydamine hydrochloride, and chamomile. Chamomile is supplied for its antiinlammatory activity whereas benzydamine hydrochloride, which has atipyrotic, antimicrobial activities, and pain relief. There is limited evidence to suggest steroids are effective. Immunomodulators are sometimes used because mucositis occurs in large part because the human immune system has been compromised during chemotherapy, leaving patients vulnerable to mucositis. Immunomodulators help strengthen the immune system, leaving patients better able to cope with mucositis. To this end, it has been shown that hemoglobin, granulocte colony stimulating factor, and Granulocyte-macrophage stimulating factor have all been shown to have some activity preventing and suppressing mucositis. Topical anesthetics, including xylocalne and linocaine rinses, are often included provide action against the pain associated with mucositis. Antiseptics, such as chlorohexidine, clean the wound and can be used as treatment and prophylaxis. Some studies suggest an inability of antiseptics to provide noticeable results against mucositis. Antibacterial agents, antiviral agents, and antifungal agents are often supplied as treatments for the components of mucositis caused by decreased immune system defenses as a result of chemotherapy and radiation therapy. Mucosal barriers and coating agents are often supplied against mucositis to protect the epidermis from infection. Cytoprotectants including, but no limited to vitamin E, are often used in the treatment of mucositis. Finally, topical analgesics are often provided to alleviate the pain associated with mucositis.

The present invention includes ingredients that include ingredients with cytoprotectant, analgesic, antiviral and mucosal barrier activity. It is, therefore, an object of the present invention to be utilized as a treatment against mucositis.

Further and other objects of the invention will be realized by those skilled in the art from the following detailed description of the invention.

This invention pertains to therapeutic composition for the prevention and treatment of HSV-1, HSV-2, zoster, aphthous ulcer, and oral and pharyngeal mucositis. This invention also pertains to methods for preparing and using the therapeutic composition and pharmaceutical products in which the therapeutical opposition may be used. Each component of the present composition has been found to provide a particular effect and the combination provides a synergistic effect that inhibits viral, bacterial, and fungal infections, heals the epidermis, diminishes epidermal damage, and minimizes pain.

The present invention is produced by mixing the ingredients until they are evenly distributed throughout the solution. The preferred embodiment of this invention contains 1% Glycyrhizic acid, 0.05% Zinc Oide, USP, 2% allantoin, 2% slippery elm, 2% Lysine (L) Monohydrochloride, USP, 2% Carnitine (L), 1% Lipoic Acid, DL-alpha (DL-thiotic acid, 0.025% Salicylic acid, USP crystalline poder, 0.5% citric acid, USP Hydrous powder, 16.25% wheat germ oil (cold pressed), 0.5% stevioside (90% extract), 4% Raspberry, 68.675% Shea butter. This disclosed formula is not intended to be limiting and the present disclosed composition may be altered to meet demands for other methods of topical delivery including but not limited mouth wash and spray delivery systems.

What is being claimed as being new and desired to be protected by Letters Patent is as follows:

1. A composition for the treatment and prevention of HSV-1 essentially consisting of glycyrrhizic acid, allantoin, slippery elm, zinc oxide lysine monohydrochloride L-carnitine, alpha-lipoic acid, salicylic acid, citric acid, vitamin E acetate, sweetener, and flavoring.

2. A composition according to claim 1 for the treatment of HSV-2.

3. A composition according to claim 1 for the treatment herpes oster, also known as shingles.

4. A composition according to claim 1 for the treatment of aphthous ulcers, also known as canker sores.

5. A composition according to claim 1 for treatment according to that supports oral and pharyngeal health in the aftermath of chemotherapy and other cancer treatments.

6. A composition according to claim 1 for the treatment of mucositis.

7. A composition according to claim 1 in which the method of delivery is a topical ointment.

8. A composition according to claim 1 in which the method of delivery is a topical spray.

9. A composition according to claim 1 in which the composition of the compound contains 1% glycyrhizic acid, 0.05% Zinc Oxide, USP, 2% allantoin, 2% slippery elm, 2% lysine (L) Monohydrochloride, USP, 2% Carnitine (L), 1% lipoic acid, DL-alpha (DL-thiotic) acid, 0.025% salicylic acid, USP crystalline poder, 0.5% citric acid, USP Hydrous powder, 16.25% wheat germ oil (cold pressed), 0.5% stevioside (90% extract), 4% raspberry, and 68.675% Shea butter,

10. A method for the prevention of herpes simplex virus with a composition of glycyrrhizic acid, inc oide, allantoin, slippery elm, Lysine (L) Monohydrochloride, carnitine (L), alpha-lipoic acid, salicylic acid, citric acid, and wheat germ oil.
